(19) United States
(12) Patent Application Publication
(10) Pub. No.: US 2019/0125727 A1
(43) Pub. Date: May 2, 2019

(54) FIXED DOSE COMBINATION FOR PAIN RELIEF WITHOUT EDEMA

(71) Applicant: Marina Biotech, Inc., Costa Mesa, CA (US)

(72) Inventor: Vuong Trieu, Agoura Hills, CA (US)

(73) Assignee: Marina Biotech, Inc., Costa Mesa, CA (US)

(21) Appl. No.: 16/173,924

(22) Filed: Oct. 29, 2018

Related U.S. Application Data
(63) Continuation of application No. 15/490,883, filed on Apr. 18, 2017, now abandoned.
Application No. 15/490,883 is a continuation-in-part of application No. 14/993,037, filed on Jan. 11, 2016, now abandoned.
Application No. 14/993,037 is a continuation-in-part of application No. 14/798,753, filed on Jul. 14, 2015, now abandoned.
Application No. 14/993,037 is a continuation-in-part of application No. PCT/US2015/011148, filed on Jan. 13, 2015.
Application No. PCT/US2015/011148 is a continuation-in-part of application No. PCT/US2015/034738, filed on Jun. 8, 2015.
Application No. PCT/US2015/034738 is a continuation-in-part of application No. PCT/US2015/034706, filed on Jun. 8, 2015.
(60) Provisional application No. 62/372,790, filed on Aug. 9, 2016.
(60) Provisional application No. 62/023,962, filed on Jul. 14, 2014.

(51) Int. Cl. A61K 31/415; A61K 31/433; A61K 45/06; A61K 31/635; A61K 31/40; A61K 31/549; A61K 31/5415; A61K 31/4155

(52) U.S. Cl. CPC A61K 31/415; A61K 31/433; A61K 45/06; A61K 31/635; A61K 31/395; A61K 31/549; A61K 31/5415; A61K 31/4155; A61K 31/40

(57) ABSTRACT

The invention provides compositions and methods for individualized therapy of arthritic pain without causing edema, using non-steroidal anti-inflammatory drugs (COX-2 inhibitor) in combination with a diuretic drug. The NSAID is preferably celecoxib and the diuretic is preferably hydrochlorothiazide in a single dose

### CROSS-REFERENCE TO RELATED APPLICATIONS

This patent application claims priority to U.S. patent application Ser. No. 15/490,883, filed Apr. 18, 2017; and U.S. Provisional Patent Application 62/372,790, filed Aug. 9, 2016 and is a Continuation-in-part application of co-pending U.S. patent application Ser. No. 14/993,037, filed Jan. 11, 2016, which is a Continuation-in-part application of co-pending U.S. patent application Ser. No. 14/798,753, filed Jul. 14, 2015, which claims the benefit of U.S. Provisional Patent Application No. 62/023,962, filed Jul. 14, 2014, and also claims the benefit of PCT Application Nos. PCT/US2015/011148, filed Jan. 13, 2015, PCT application No. PCT/US2015/034738, filed Jun. 8, 2015, and PCT/US2015/034706, filed Jun. 8, 2015, all seven of which are incorporated by reference, the entire disclosures of which are hereby incorporated by reference.

### BACKGROUND OF THE INVENTION

It is well appreciated that non-steroidal anti-inflammatory drugs (“NSAID”) are highly active analgesics. However, NSAIDs also can have clinically significant side effects. One such side effects is drug induced edema.

“Edema” is an abnormal accumulation of fluid in the tissue spaces, cavities, or joint capsules of the body, causing swelling of the area. Edema can occur in the tissues or body spaces such as the plural cavities or the peritoneal space. Clinically, edema has variable consequences depending on the site and severity of the edema. In contrast, chronic, severe subdermal edema can cause skin break down, ulceration and serious infection. Similarly, while a pleural effusion may spontaneously resolve, ascites (edema in the peritoneal space) can be complicated by difficult to treat bacterial peritonitis. See, e.g., Harrison's Internal Medicine, 16th edition, p.213-214.

The causes of edema are often complex. Pathophysiologically, edema occurs when one or more of the following is present: elevated capillary hydraulic pressure, increased capillary permeability an when the interstitial oncotic pressure exceeds the plasma oncotic pressure. Such changes can result from a variety of conditions and diseases. For example, in congestive heart failure the activation of the renin-angiotensin system causes volume overload which results in increased capillary hydraulic pressure. The kidneys control extracellular fluid volume by adjusting sodium and water excretion. When renal function is impaired, edema can result. In cirrhosis the reduced production of serum proteins such as albumin result in a decrease in the plasma oncotic pressure relative to interstitial oncotic pressure resulting fluid shifts into the interstitial space. Venous insufficiency is a common cause of edema of the lower extremities from an increase in capillary hydraulic pressure. See, e.g., Harrison's Internal Medicine, 16th edition, p.213-214; O'Brian et al., “Treatment of Edema,” American Family Physician, 71(11). 2111-17.

Many drugs can cause edema including, without limitation, steroid hormones, vasodilators such as hydralazine, estrogens, NSAIDs, immunomodulators such as interleukin 2, and calcium channel blockers. Like other forms of edema, the pathophysiology of drug induced edema is wide ranging. Drug induced edema may be caused by vasodilation (e.g. hydralazine), drug effects on the kidneys' sodium excretion (e.g., steroids), and capillary damage (e.g., interleukin 2). Drug induced edema is usually dose-dependent and its severity increases over time. Harrison's Internal Medicine, 16th edition, p.213-214; O'Brian et al., Treatment of Edema, American Family Physician, 71(11): 2111-17. Many NSAIDs can cause edema. The mechanism for NSAID induced edema has been postulated to be from renal vasoconstriction. Harrison's Internal Medicine, 16th edition, p.213-214.

NSAIDs inhibit cyclogenases (COX), the enzymes that catalyzes formation of various prostaglandins. The two principle COX isoforms are COX-1 and COX-2. Studies have shown that both therapeutic and side effects of NSAIDs are dependent on cyclooxygenase inhibition. In general, selective inhibitors of COX-2 have therapeutic effects that are as strong as conventional NSAIDs but with fewer side effects. Nevertheless, selective COX-2 inhibitors still can cause edema. Heyman et al., Anti-inflammatory and side effects of cyclooxygenase inhibitors, Pharma. Reports, 2007 59:247-258.

Any suitable means may be used in the detection and quantification edema varies widely. For example, effusions (edema in the thoracic, peritoneal, or pericardium) can be quantified based on the level of fluid when imaged with the patients standing. Most commonly, edema is measured subjectively based on the ability to push into or “pit” the swollen skin.

Celecoxib (under the brand name “CELEBREX®”) is a prototypic selective COX-2 inhibitor and the first page of the CELEBREX® Package Insert lists edema as an “adverse reaction.” Table 1 of this Package Insert discloses that 2.1% of patients treated with celecoxib develop edema, as compared to 1.1%, 2.1%, 1.0%, and 3.5% for placebo, naproxen, diclofenac, and ibuprofen, respectively. Moore et al.'s review of the tolerability and rate of adverse events in clinical trials of celecoxib found that the incidence of edema at any site was usually about 3%, but in two trials the incidence of edema was 23% and 38%. Arthritis Res. & Therapy, 2005, 7(6), R644-R664, R658-59 MV.

Treatment of edema consists of reversing the underlying disorder (if possible), restricting dietary sodium to minimize fluid retention, and, usually, employing a diuretic drug. O'Brian et al., Treatment of Edema, American Family Physician, 71(11): 2111-17.

In view of the persistent problem of drug induced edema and, in particular, edema induced by drugs with known efficacy for the treatment of pain, there remains a need for better approaches to preventing and treating drug induced edema. In addition, despite progress in the art, because each of the multiple mechanisms that produce drug induced edema require a specialized treatment, there remains a need for better approaches to preventing and treating drug induced edema.

### BRIEF SUMMARY OF THE INVENTION

In view of the issues set forth above, the invention provides compositions for treating pain without inducing edema comprising a NSAID, preferably a COX-2 inhibitor, most preferably celecoxib, and a diuretic drug, preferably a diuretic, most preferably HCTZ, wherein the composition is administered in a fixed-dose combination (FDC) of the NSAID and the diuretic. The preferred embodiment is celecoxib/HCTZ FDC. This was from our discovery that patient treated simultaneously with celecoxib and HCTZ have significantly lower incidence of edema than patient treated with celecoxib and any other drugs. HCTZ was selected since we found that non-Thiazide diuretic actually caused an increase in edema when combined with Celebrex.

During our development of celecoxib/HCTZ FDC, we found that celecoxib suppressed the dissolution of HCTZ during dissolution studies and simple mixture of the two drug substance to make an FDC is not acceptable. The two components have to be formulated in separate compartments and excipients of the HCTZ component have to be adjusted to compensate for the suppression of dissolution by celecoxib.

Additionally, provided is a method for individualized therapy of pain without inducing edema using a NSAID including wherein the NSAID may be a COX-2 inhibitor and in a preferred embodiment the NSAID is celecoxib. This application seeks the treatment of patients at target celecoxib AUC of 3400 ng*hr/mL (Mean AUC of 100 mg dose) or celecoxib AUC of 6800 ng*hr/mL (Mean AUC of 200 mg dose). Celecoxib AUC can be determined for example by LC/MS method with blood samples at various time points over a 48 hr period. There is no known method of predicting celecoxib AUC—therefore actual celecoxib AUC determination for each patient is required.

This invention is the AUC dosing of celecoxib/HCTZ for the optimum treatment of osteoarthritis pain without increase risk of edema from celecoxib. The preferred embodiment is celecoxib/HCTZ formulated as either pill in pill, capsule in capsule, bilayer tablet or other formulation method with physical separation between celecoxib and HCTZ. Additionally, the HCTZ excipients were designed to counter the suppression of HCTZ dissolution by celecoxib.

### BRIEF DESCRIPTION OF THE DRAWINGS

FIG. 1 is a scatterplot graph displaying the relationship between LPS-stimulated plasma PGE2 ex vivo, an index of NSAID activity, and log plasma concentrations of celecoxib 2, 4, 6, and 24 hours after dosing. PGE2 is expressed as a percentage of pre-dosing values. A steep but variable dose-response is evident. (P, 0.01 vs. placebo) (from McAdam et al. Systemic biosynthesis of prostacyclin by cyclooxygenase (COX)-2: the human pharmacology of a selective inhibitor of COX-2. PNAS. 1999; 96:272-7.)

FIG. 2 depicts the pharmacokinetic parameters produced by different doses of celecoxib.

FIG. 3 displays the result of a meta-analysis of the one dose AUC from patients in different age groups.

FIG. 4 displays the result of a meta-analysis of the celecoxib dose dependence of edema.

FIG. 5 displays the result of a regression analysis of the celecoxib dose dependence of edema.

FIGS. 6-11 display the incidence of edema for celecoxib given alone or with other drugs. HCTZ, hydrochlorothiazide; CCB, calcium channel blocker; ARB, angiotensin receptor blocker; ACE, angiotensin converting enzyme inhibitor; non-thiazide diuretic; beta blocker; Any drug other than celecoxib.

FIGS. 12 and 13 depict the dissolution profiles from a FDC.

FIG. 14 depicts dissolution profiles for HCTZ+celecoxib filled as a mixture or filled separately.

FIG. 15 shows dissolution profiles for HTCZ+celecoxib in a bilayer tablet.

FIG. 16 shows dissolution profiles for HTCZ+celecoxib in a tablet plus powder in a capsule.

FIG. 17 shows dissolution profiles for HTCZ+celecoxib in a powder plus powder in a capsule.

FIG. 18 shows dissolution profiles for HTCZ+celecoxib in a capsule in a capsule.

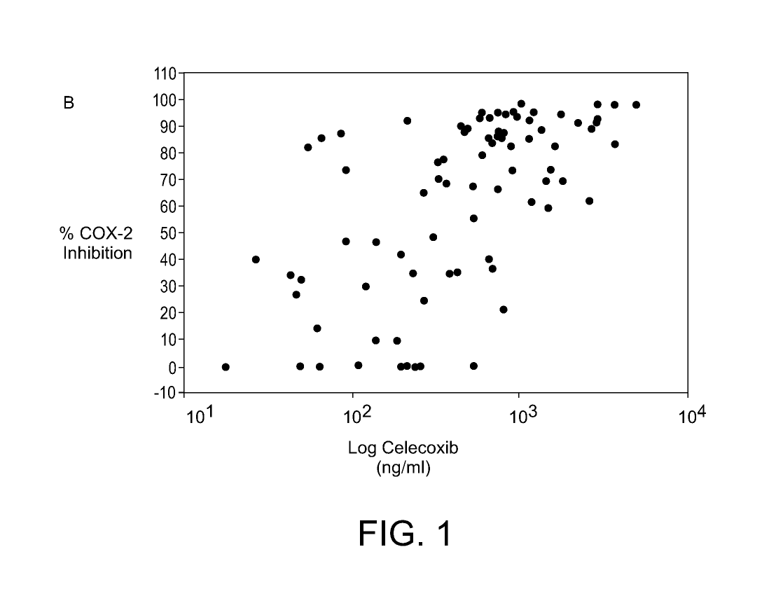

### DETAILED DESCRIPTION OF THE INVENTION

In one aspect, the invention provides methods for treating osteoarthritis pain with a COX-2 inhibitor, in general, and celecoxib or celecoxib/HCTZ FDC, in specific. In the method, a dosing regimen targeting specific AUC is provided in which AUC determined from first dosing with celecoxib is used to adjust subsequent dosing to achieve the targeted AUC. The targeted AUC dosing regimen for celecoxib was made possible by our discovery of: 1) the targeted AUC value derived from our analysis of celecoxib pharmacokinetics; and 2) the method of adjustment taking advantage of our demonstration of celecoxib dose proportionality.

In certain embodiments, the pharmacokinetic parameter used in the method is area-under-the-curve (AUC).

In certain embodiments, the therapeutic agent is celecoxib/HCTZ FDC.

The methods of the invention are effective in treating osteoarthritis pain.

As noted above, antihypertensive drug pharmacokinetic variability results in variability in celecoxib therapy effectiveness. In the methods of the present invention, determination of drug exposure as AUC allows for adjustment of subsequent dosing because of the observed linear correlation was found between celecoxib drug dose and the AUC.

The invention provides compositions and methods for individualized therapy of pain, including but not limited to arthritic pain, using a NSAID, preferably a COX-2 inhibitor, more preferably the COX-2 inhibitor celecoxib and an antihypertensive drug, such as a diuretic (e.g., hydrochlorothiazide). The high patient to patient variability in response to a dose of any NSAID and/or antihypertensive drug makes the mere clinical monitoring of patients an inadequate way to treat patients with this class of drugs. Accordingly, the measurement of “blood levels” (i.e., the occasional measurement of the drug's concentration in the plasma or serum) is unlikely to lead to effective nontoxic regimens. Given the complexity of NSAIDs' dose response relationships both alone and in combination with one or more drugs, a more comprehensive set of metrics must be employed in each patient.

The methods claimed herein take advantage of a pharmacokinetic (“PK”) analysis for each patient. As such, the claimed methods go beyond the measurement of a single blood level at a single time point. Instead, the claimed methods make use of data on the plasma drug concentration from several time points (at least 2, preferably at least 5, 6, 7, 8, 9, 10, 11, 12 over period of at least 12, 18, 24, 36, 48, 60, 72 hours) and take advantage of the full scope of PK parameters to generate a PK “profile” unique to a given patient for a particular drug. There is no known method of predicting individual PK profile for celecoxib due to the complexity of human pharmacokinetics. Significantly, there is no natural law known that can predict human pharmacokinetics without administering at least one dose of given drug to an individual. In addition, the multitude of potential contributing factors make defining such a law impractical. Therefore, the methods disclosed herein seek to determine the individual's PK profile directly.

As used herein “pain” refers to physical suffering or discomfort caused by an illness or injury, e.g., arthritis.

As used herein “osteoarthritic pain” refers to pain resulting from, e.g. osteoarthritis (aka, “degenerative joint disease”).

As used herein, “formulation” or “a formulation” refers to a combination of active ingredients and pharmaceutically acceptable carriers wherein each is present in a dosage form at fixed ratios to one another (i.e., fixed percentages of each ingredient in the dosage form.)

As used herein, the phrase “COX-2 inhibitor concentration/time data points” refers to the COX-2 inhibitor and diuretic concentration in a unit of volume (e.g., 1 ml) of plasma from a subject at a given point in time before or after administration of the COX-2 inhibitor and diuretic.

As used herein, the phrase “transforming” the patient's COX-2 inhibitor and the antihypertensive drug “concentration/time data points” refers to the application of mathematical operations, formulas, theories, and/or principles to the COX-2 inhibitor or diuretic concentrations/time data points of an individual to derive PK parameters (e.g., a formula for calculating AUC).

As used herein, “trace edema” is edema that is just above the threshold for detection on physical exam (inconsistently pitting) and does not significantly impair the patient's functioning in society or the patient's physiologic functions.

As used herein, “pain control is adequate” refers to a level of pain the patient is willing to live with and which does not significantly impair the patient's functioning in society or the patient's physiologic functions.

As used herein, “toxicity is acceptable” refers to the absence of significant side effects and a level of toxicity that the patient is willing to live with and does not significantly impair the patient's functioning in society or the patient's physiologic functions.

As used herein, “NSAID” or “non-steroidal anti-inflammatory drug” refers to a class of drugs which provide pain-reducing and fever reducing effects as well as anti-inflammatory effects in a subject, e.g., a human patient. NSAIDs include both COX-1 and COX-2 inhibitors.

As used herein, a “COX-1 inhibitor” refers to a non-steroidal anti-inflammatory drug that is capable of directly targeting the COX-1 enzyme in a subject and inhibits at least some COX-1 activity, e.g., aspirin.

As used herein, a “COX-2 inhibitor” refers to a non-steroidal anti-inflammatory drug that is capable of directly targeting the COX-2 enzyme in a subject and inhibits at least some COX-1 activity, e.g., celecoxib. As used herein, a “mixed COX-1 and COX-2 inhibitor” refers to a non-steroidal anti-inflammatory drug that is capable of directly targeting both the COX-1 and COX-2 enzymes in a subject and inhibits at least some COX-1 and COX-2 activity, e.g., ibuprofen.

As used herein the term “diuretic” refers to any substance that promotes the production of urine. A diuretic may also exhibit an antihypertensive action. Suitable diuretics for use in the compositions and methods disclosed herein include but are not limited to amiloride (MIDAMOR®), bumetanide (BUMEX®), chlorothalidone (HYGROTON®), ethacrynic acid (EDECRIN®), furosemide (LASIX®), hydrochlorothiazide (DIURIL®), indapamide (LOZOLV®), metolazone (ZAROXOLYN®), torsemide (DEMADEX®), triamterene, acetazolamide, theophylline, chlorthalidone, spironolactone, and combinations thereof.

As used here the combination can also include a “fixed dose combination” (FDC). These fixed dose combinations can be in the form of pill in pill, capsule in capsule, bilayer tablet or other formulation method with physical separation between celecoxib and HCTZ.

The invention provides compositions and methods for individualized therapy of pain, including but not limited to arthritic pain, using a non-steroidal anti-inflammatory drug (NSAID), preferably celecoxib in combination with a diuretic, preferably hydrochlorothiazide. In addition, the invention provides methods for predicting the outcome of the therapy of pain with a composition comprising NSAID, preferably a COX-2 inhibitor, most preferably celecoxib, and a diuretic, preferably hydrochlorothiazide. Further, the invention provides methods of using a NSAID, preferably celecoxib, and a diuretic, preferably hydrochlorothiazide, in the manufacture of medicament for the treatment of pain.

In one embodiment, provided is a composition for treating pain without inducing edema comprising a NSAID and a diuretic, wherein the composition is administered in a fixed-dose combination. Any type of pain may be treated by the composition, including arthritic and osteoarthritic pain.

The NSAID may be one or more of the following NSAIDs, but is not limited thereto: diclofenac, diflusnisal, etodolac, fenoprofen, flubiprofen, ibuprofen, indomethacin, ketoprofen, ketorolac, meclofenamate, nabumetone, naproxen, oxaprozin, phenylbutazone, piroxicam, salicylate, sulindac, tolmetin, celecoxib, rofecoxib, etoricoxib, lumiracoxib, parecoxib, valdecoxib, chlorthalidone and combinations thereof. In a preferred embodiment, the COX-2 inhibitor is celecoxib.

The diuretic may be one or more of the following diuretics: amiloride, bumetanide, chlorothalidone, ethacrynic acid, furosemide, hydrochlorothiazide, indapamide, metolazone, torsemide, triamterene, acetazolamide, theophylline, chlorthalidone, spironolactone, and combinations thereof. In a preferred embodiment, the diuretic is hydrochlorothiazide.

The combination can include a “fixed dose combination” (FDC). These fixed dose combinations can be in the form of pill in pill, capsule in capsule, bilayer tablet or other formulation method with physical separation between celecoxib and HCTZ. The composition may be administered, for example, daily, twice a day, three times a day, four times a day, or every other day.

One or more pharmaceutically acceptable carriers or excipients may be included in the composition. Any suitable pharmaceutical carrier can be used in accordance with the invention. Suitable pharmaceutical carriers include, without limitation, sterile water, saline, dextrose, dextrose in water or saline, sium or calcium stearate and/or polyethylene glycols, arabic gums, gelatin, methyl cellulose, carboxymethylcellulose, hydroxypropylmethylcellulose, ethyl cellulose, cellulose, crospovidone, povidone, acrylic and methacrylic acid co-polymers, pharmaceutical glaze, gums, solvents, ethanol, isopropyl alcohol, methylene chloride or sugar, lactose, gelatin, starch, silicon dioxide, diethyl phthalate, diethyl sebacate, triethyl citrate, cronotic acid, propylene glycol, butyl phthalate, dibutyl sebacate, castor oil, diethyl phthalate, diethyl sebacate, lactose, dextrose, saccharose, cellulose, starch or calcium phosphate, olive oil or ethyl oleate silica, talc, stearic acid, magnesium or calcium stearate,polyethylene glycols; clays, gum tragacanth or sodium alginate, binding agents such as starches, arabic gums, polyvinylpyrrolidone, alginic acid, sodium starch glycolate, polysorbates, laurylsulphates; and other therapeutically acceptable accessory ingredients, such as humectants, preservatives, buffers and antioxidants, which are known additives for such formulations, lactose, dextrose, saccharose, cellulose, ethyl oleat, talc, stearic acid, magnesium or calcium stearate and/or polyethylene glycols, arabic gums, gelatin, methylcellulose, carboxymethylcellulose, polysorbates and combinations thereof.

It should be understood that in addition to the ingredients particularly mentioned above, the formulations of this invention can include other suitable agents such as flavoring agents, preservatives and antioxidants. Such antioxidants would be food acceptable and could include vitamin E, carotene, BHT or other antioxidants known to those of skill in the art.

The composition may be administered in a fixed dose combination, for example, without limitation, wherein the NSAID and the diuretic are at the following strengths (celecoxib/hydrochlorothiazide) 100 mg/12.5 mg, 200 mg/12.5 mg, 100 mg/25 mg, 200 mg/25 mg.

The composition may include any suitable NSAID and diuretic dosage. For example, without limitation, the composition may comprise 50 to 400 mg, 75 to 350 mg, 100 to 300 mg, 150 to 250 mg, 50 mg, 100 mg, 200 mg, or 400 mg of NSAID. The composition may comprise 25 to 200 mg, 50 to 150 mg, 75 to 100 mg, 50 mg, 100 mg, 150 mg, or 200 mg of diuretic.

The composition may be administered to any suitable subject, including mammals. Suitable mammals include but are not limited to humans.

Any suitable NSAID can be used in accordance with the invention, including without limitation, a COX-1-specific inhibitor, a COX-2-specific inhibitor, a mixed COX-1 and 2 inhibitor or a combination thereof. As such, the NSAID can be a salicylate, propionic acid derivative, acetic acid derivative, enolic acid derivative, anthranilic acid derivative or combinations thereof. Accordingly, the NSAID can be, aspirin (acetylsalicylic acid), ibuprofen, naproxen, indomethacin, sulindac, piroxicam, clonixin, preferably celecoxib or a combination thereof. In addition, the invention can be used with combinations of NSAIDs and other analgesic drugs such as lidocaine, opiates, acetaminophen, tricylic antidepressants, anticonvulsants, carbamazepine, gabapentin, and pregabalin; other anti-inflammatory drugs such as steroids and immunosuppressants. Further, the invention can be used with combinations of NSAIDs and other therapies for arthritis, including but not limited to, methotrexate and gold-salts.

The following examples further illustrate the invention but, of course, should not be construed as in any way limiting its scope.

### EXAMPLES

### Example 1

The approved prescribing information for CELEBREX® (celecoxib) as listed on its package insert (for US/EU/ROW) instructs that a physician should use lowest effective dose for the shortest duration consistent with treatment goals for the individual patient. For four of the six approved indications the package insert includes a 100 mg BID regimen:

1) Osteoarthritis (OA): 200 mg QD or 100 mg BID.

2) Rheumatoid Arthritis (RA): 100 mg BID or 200 mg BID.

3) Juvenile Rheumatoid Arthritis (JRA): 50 mg BID in patients 10-25 kg. 100 mg BID in patients more than 25 kg.

4) Ankylosing Spondylitis (AS): 200 mg once daily single dose or 100 mg BID.

5) Acute Pain (AP) and 5) Primary Dysmenorrhea (PD). 400 mg initially, followed by 200 mg dose if needed on first day. On subsequent days, 200 mg BID as needed.

Unexpectedly, however, the inventor's analysis of the actual prescribing behavior using Evaluate Pharma/IMS database determined that the 200 mg is the predominant dose being prescribed by physicians by more than 10 to 1. These data are consistent with data from a MEPS survey (Table 1) and Medicaid survey (Table 2). In view of predominance of the 200 mg dosage form sales and the evidence that the 100 mg and 200 mg doses produce overlapping PK and pharmacodynamic results, it is questionable that the package insert's admonition that “the lowest dose of CELEBREX® should be sought for each patient” is followed. Instead, the data indicates that it is likely that there are numerous patients at risk for ADRs because their celecoxib dose is higher than it needs to be (i.e., 200 mg BID rather than 100 mg BID).

TABLE 1
MEPS Survey Data

| | | USA | USA |
|---|---|---|---|
| | Strength | sales | sales |
| Package description | (mg) | 2008 | 2011 |
| 100 capsule in bottle (0025-1525-31) | 200 | 1,553 | 1,650 |
| 500 capsule in 1 bottle (0025-1525-51) | 200 | 323 | 132 |
| 100 capsule in 1 bottle (0025-1520-31) | 100 | 65 | 142 |
| 500 capsule in 1 bottle (0025-1520-51) | 100 | — | 17 |
| 100 blister pack in 1 carton (0025-1520- | 100 | — | 7 |
| 34) >1 capsule in 1 blister pack | | | |
| 120 capsule in 1 bottle (63629-3021-5) | 200 | — | 2 |
| 30 capsule in I bottle, plastic (67544-204-30) | 200 | — | 32 |
| Total | | 1,989 | 1,982 |

TABLE 2
Prescribing information derived from Medicaid

| Dose | Number of | Number of | Number of | Number of | Number of | Number of | Number of |
|---|---|---|---|---|---|---|---|
| (mg) | RXs 2008 | RXs 2009 | RXs 2010 | RXs 2011 | RXs 2012 | RXs 2013 | RXs 2014 |
| 50 | 293 | 485 | 856 | 1169 | 1167 | 1396 | 726 |
| 100 | 33,436 | 36,023 | 43,755 | 47,524 | 35,399 | 34,178 | 16,628 |
| 200 | 320,628 | 330,521 | 380,546 | 384,404 | 285,764 | 250,985 | 114,532 |
| 400 | 1,383 | 1,636 | 3,116 | 3,476 | 2,565 | 2,240 | 1,093 |
| Total | 355,740 | 368,665 | 428,273 | 436,573 | 324,895 | 288,799 | 132,979 |

### Example 2

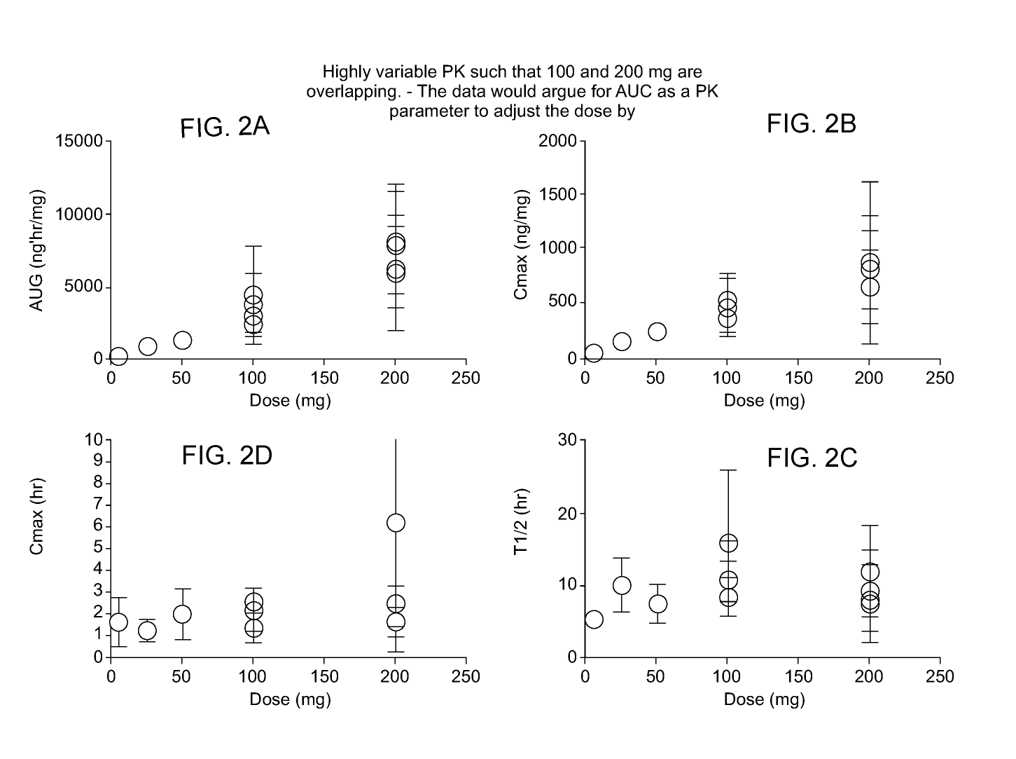

The combined plots of published pharmacokinetic data including those from the Summary basis for approval are shown in FIG. 2. The variability of CELEBREX® pharmacokinetics were unexpectedly high. The PK results for the 200 mg dose shows a substantial overlap with that of the 100 mg dose. Accordingly, the dose proportionality may not be as is described by the package insert for CELEBREX®. As a result of the failure to determine and pursue target PK ranges, in some instances patients receiving 100 mg patients may not get enough of the drug and the 200 mg patients may receive too much of the drug.

### Example 3

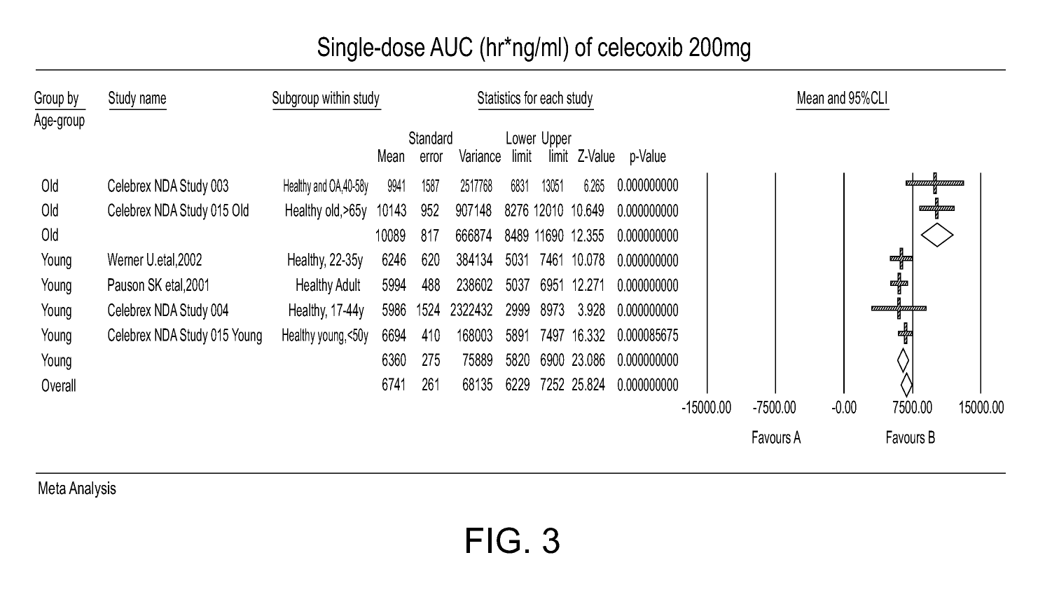

Applicant's meta-analysis of the reported PK parameters in different populations demonstrates that the elderly show a higher variability than younger patients. For example, when the applicant's meta-analysis is presented in age-based subgroups, the elderly and younger patients demonstrate highly significant differences in drug exposure as defined by AUC (FIG. 3). In other words, the most efficacious celecoxib dosage is not well defined among the elderly. The problem may be more widespread than expected as elderly here is defined as patients greater than >40 or >50, not the usually definition of elderly (age greater>65). Previously, there has been reported impaired PK with elderly and the package insert issued warning on impaired PK in elderly but did not suggest dose reduction. Our finding suggests that the issue is more substantial and more widespread and also includes middle aged groups.

There is variability in PK results within groups and the Cₘₐₓ and AUC overlap between the 100 mg and 200 mg groups indicates that correctly dosing elderly patients to maximize celecoxib efficacy at the lowest doses possible depends on many individualized, unpredictable variables. Based on the wide range of AUC values, some patients receiving 100 mg may not get enough of the drug and the 200 mg patients may receive too much of the drug (FIG. 2).

### Example 4

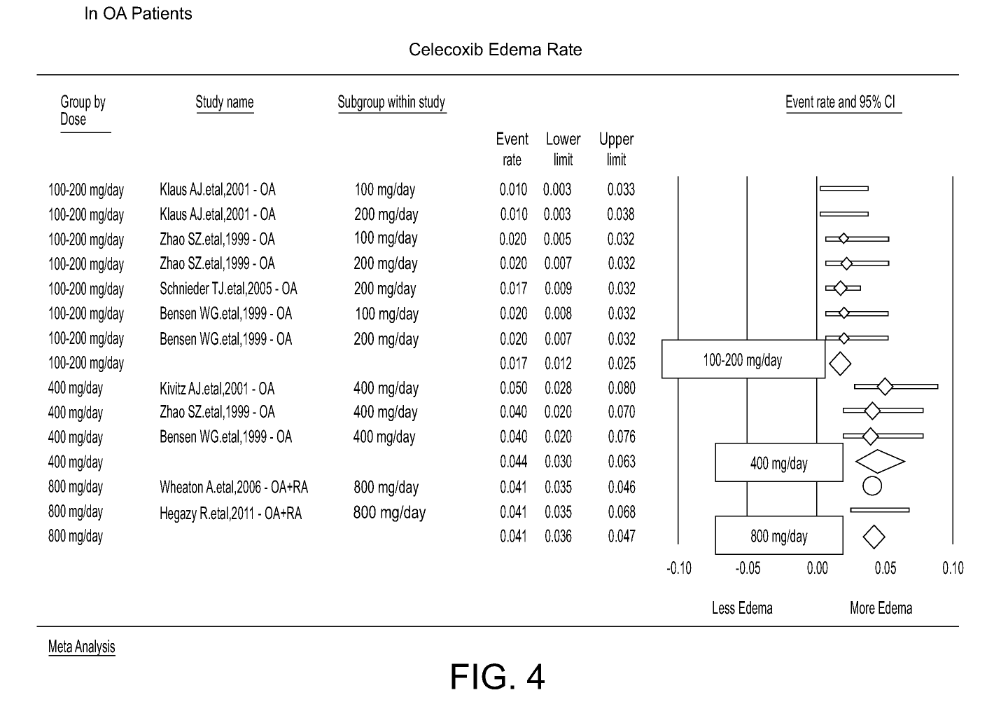

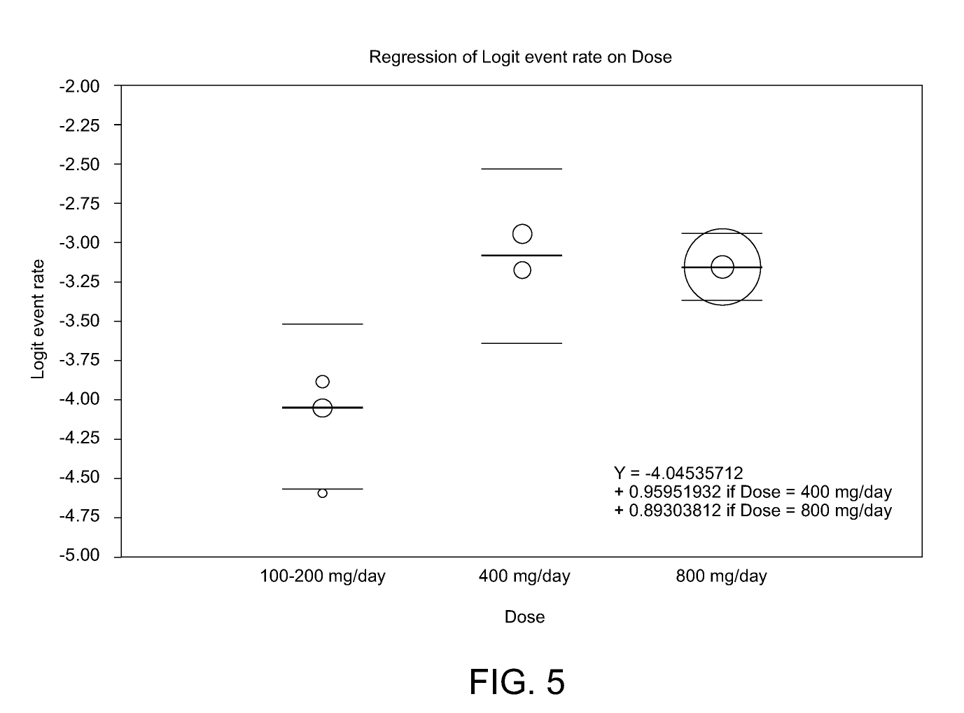

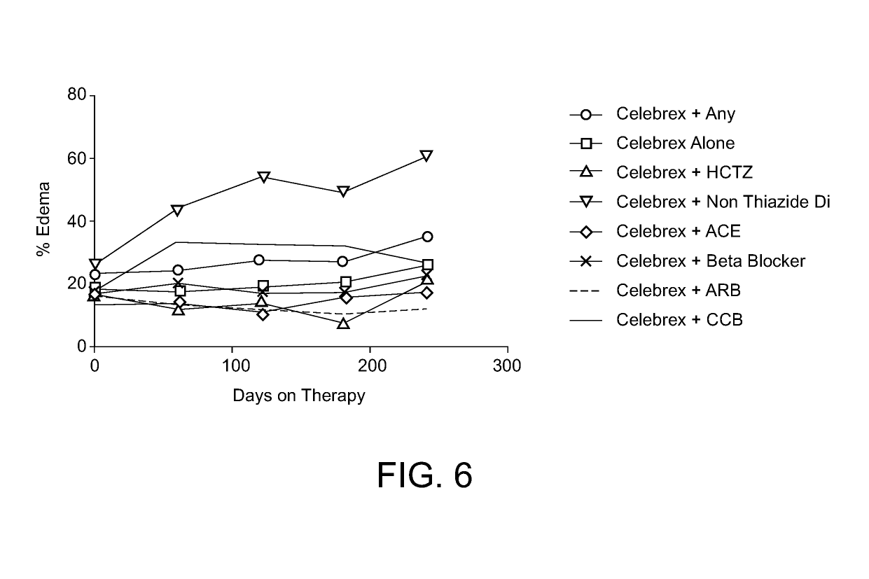

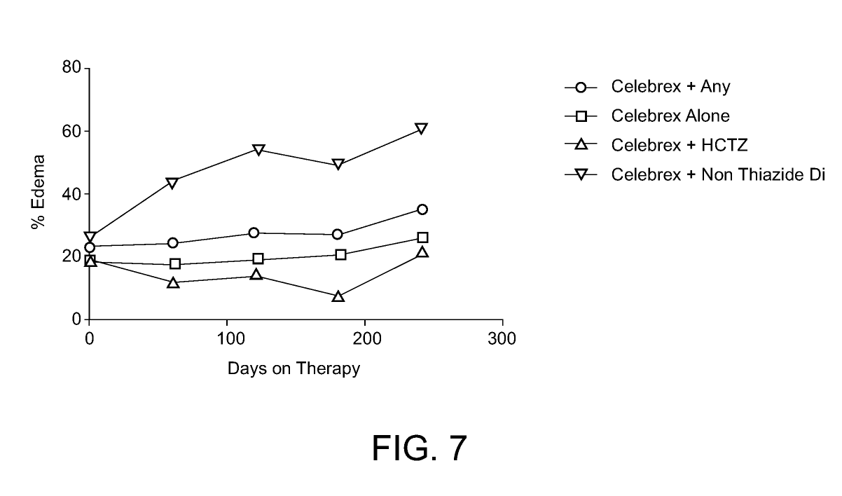

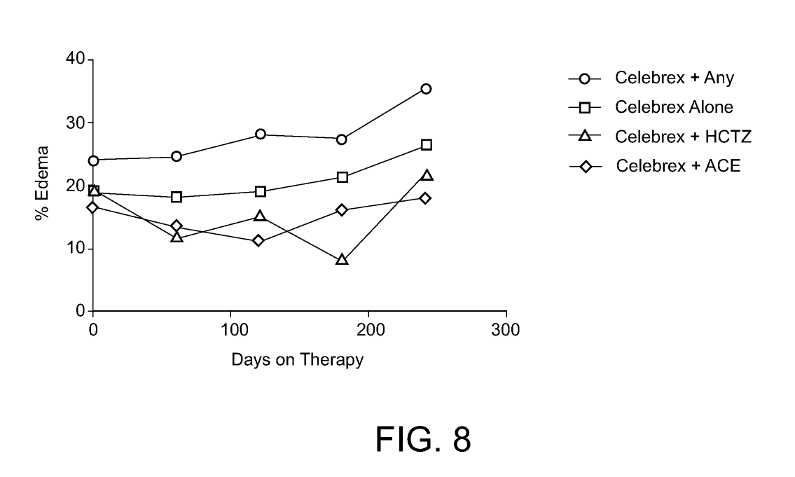

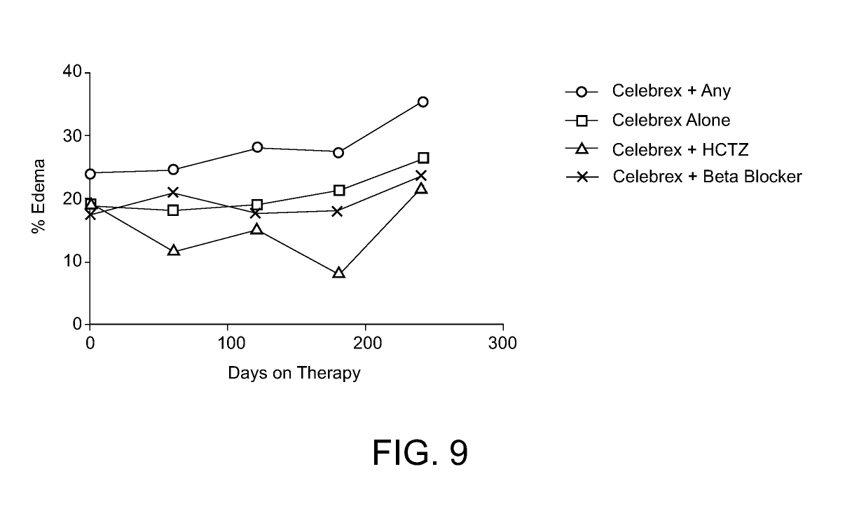

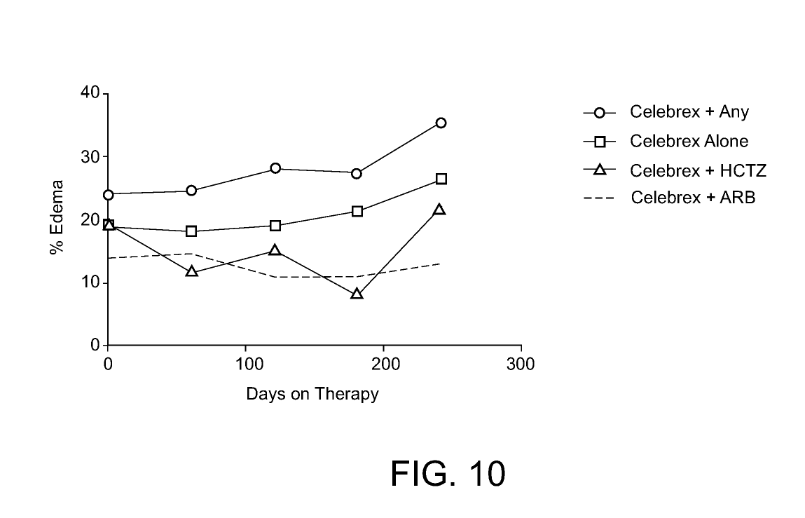

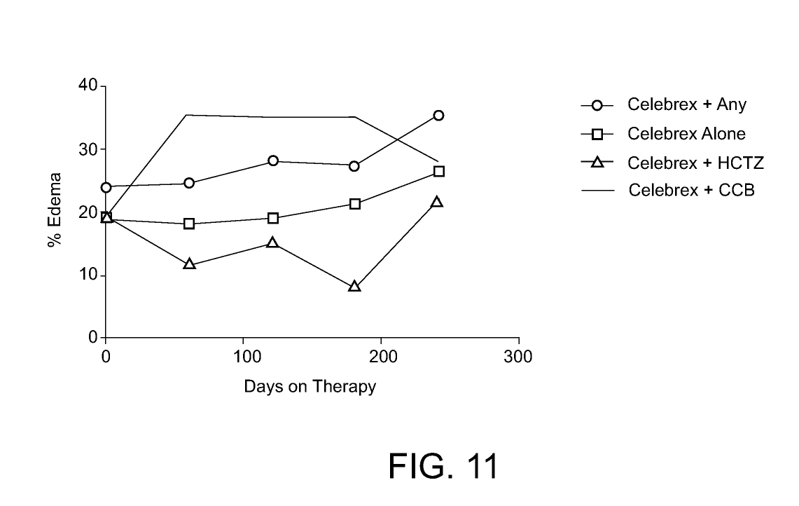

Applicant's meta-analysis of the reported edema rates in different osteoarthritic populations receiving doses of celecoxib ranging from 100 mg/day to 800 mg/day reveals that edema event rates are significantly higher in osteoarthritic populations receiving doses of celecoxib that are greater than 200 mg/day. For example, both the upper limit of the edema event rate and the average edema event rate in osteoarthritic populations receiving a 400 mg/day dose of celecoxib can be more than twice as high as the upper limit edema event rate and the average edema event rate seen in osteoarthritic populations receiving a 100 or 200 mg/day dose of celecoxib (FIGS. 4-5). Additionally, Applicant's meta-analysis of the reported edema rates in different osteoarthritic populations receiving doses of celecoxib ranging from 100 mg/day to 800 mg/day reveals that patients receiving a 100 or 200 mg/day dose of celecoxib experience remarkably similar edema event rates. This analysis indicates that a patient who is selected to receive a 200 mg/day dose of celecoxib based on, for example, their individual pharmacokinetic data using one or more of the methods described herein, will not be at a higher risk for an edema event than a patient receiving a 100 mg/day dose of celecoxib, and vice versa.

### Example 5

Applicant has also compared the edema in patient populations receiving celecoxib alone, celecoxib in combination with a variety of antihypertensives, including thiazides, celecoxib in combination with hydrochlorothiazide, celecoxib with a non-thiazide diuretic and no anti-hypertensives, and celecoxib with a non-thiazide anti-hypertensive. To support this study a database was created which contains: 1) Claims data from SYMPHONY DATABASE® pertaining to anti-hypertensives, Statins, COX-2's, and NSAIDS. The data span the most recent 36 months and 2) registry data from the ACC reporting Blood Pressure (systolic/diastolic), peripheral edema flags (yes, no, missing), Heart rate, LDL, Glucose Level, Ejection fraction, GFR, Height, Weight, BJVII, and the like.

SYMPHONY DATABASE® contains true patient level data—All Data Sources be it RX or JVIX claims is tied back to individual patients which is tracked and then encrypted based on first name, last name, gender, DOB and zip code to give an accurate picture of patient level informatics year over year regardless of insurance changes. The source of Managed Markets Rx claims data comes from various providers, including Intelligent network services (Switch Data) as well as direct data feeds from pharmacies that do not use Switches so it does not create payer biases.

The properties for the SYMPHONY DATABASE® are: 1) Takes CELEBREX® antihypertensive, AH, Statin or NSAID or have OA, RA or some other form of arthritis, 36 months, 2) Time Frame=Jan. 1, 2012-Dec. 31, 2014 (3 years), 3) Number of files=201, 4) Size=561 GB zipped (−2.5 TB), 5) Unique Patients=162 million, 6) Patients on CELEBREX®=4.3 million, 7) Patients that have OA=16.3 million (15.4 million only OA), 8) Patients that have RA=2.3 million (1.4 million only RA).

The properties of the ACC registry are: 1) Have 3+BP readings, 2) Time Frame=Jan. 1, 2012-Dec. 31, 2014 (3 years), 3) Number of files=2, 4) Size=590 MB, 51 MB, 5) Unique Patients=1.58 million, 6) Patients with BP readings=1.58 million, 7) Patients with Edema Flag True=870 K.

The edema rate was then measured in the aforementioned database. The incidence of edema was higher for OA patients than RA, other Arthritis, or Arthritis free patients. The incidence of edema increased when patients was taken CELEBREX® for all groups except for RA and no Arthritis free patients. Overall OA seems to be susceptible to CELEBREX® induced edema. The results confirmed the meta-analysis shown above.

TABLE 3
Shows cases of edema (%) among OA, RA, other arthritis, and no arthritis

| Group | OA | | RA | | Other Arthritis | | No Arthritis | |
|---|---|---|---|---|---|---|---|---|
| Wlo CELEBREX ® | 12,150 | (4.7%) | 3,337 | (3.0%) | 164,837 | (2.3%) | 69 | (0.4%) |
| WI CELEBREX ® | 254 | (6.0%) | 82 | (3.3%) | 1702 | (4.1%) | 451 | (0.4%) |

### Example 6

The same database in Example 5 was used to determine whether the thiazide diuretic hydrochlorothiazide (HCTZ) would reduce the incidence of edema in patients taking CELEBREX®. There was a steady increase in incidence of edema among patient taking CELEBREX® only. This trend was exacerbated by additional Rx. The incidence was lower for patients taking CELEBREX® and HCTZ. In contrast, the incidence of patients taking CELEBREX ® and non-thiazide diuretics resulted in more than doubling of the incidence of edema. The data is surprising in that edema can only be controlled selectively by HCTZ (a thiazide diuretic) and not by other non-thiazide diuretics. The non-thiazide diuretics include: 1) Loop: torsemide, furosemide, bumetanide, ethancrynic acid, 2) Carbonic Anhydrase Inhibitors: acetazolamide, dichlorphenamide, methazolamide, 3) Potassium sparring: triamterene, spironolactone, amiloride, and 4) Others: pamabrom, and mannitol.

| | | | | | | | CELEBREX ® + | |
|---|---|---|---|---|---|---|---|---|
| | CELEBREX ® + | | CELEBREX ® | | CELEBREX ® + | | non-thiazide | |
| Days of Therapy | Any Rx | | Only | | HCTZ | | Diuretics | |
| <60 | 139,389 | (23.9%) | 20,663 | (19.4%) | 455 | (19.1%) | 2146 | (26.7%) |
| 60-120 | 14,361 | (24.7%) | 913 | (18.1%) | 50 | (12.0%) | 160 | (44.4%) |
| 120-180 | 12,272 | (28.2%) | 678 | (19.3%) | 33 | (15.2%) | 125 | (54.4%) |
| 180-240 | 12,620 | (27.6%) | 661 | (21.6%) | 47 | (8.5%) | 49 | (49.0%) |
| >240 | 55,396 | (35.7%) | 1,991 | (26.7%) | 77 | (22.1%) | 453 | (60.9%) |

### Example 7

The frequency of patients experiencing edema was examined. Two groups were used: those taking CELEBREX® for any length of time and those taking CELEBREX® for more than 180 days. As shown below, those taking CELEBREX® for greater than 180 days experienced higher incidence of edema. In both cases, combining CELEBREX® with HCTZ effectively inhibited CELEBREX® induced edema significantly. This was more evident when comparing the CELEBREX®+ HCTZ inhibitor versus CELEBREX®+ any drug regardless of class. The results are surprising as addition of another drug to CELEBREX® regime would be expected to induced additional drug induced toxicity. Indeed, addition of non-thiazide anti-hypertensive to CELEBREX® significantly worsens the incidence of edema. Therefore we unexpectedly found here that not just any combination of CELEBREX® to a diuretic would work, it has to be HCTZ and not any of the non-thiazide diuretics.

Table 5 shows incidence of edema when patients taking CELEBREX® alone, CELEBREX® plus any other drug, CELEBREX® and various classes of anti-hypertensive drugs. For this group, the patients are on CELEBREX® for any length of time.

TABLE 5
Incidence of edema, all patients on celecoxib

| #Pts | Edema | No Edema | Total Pts # | % Edema |
|---|---|---|---|---|
| Alone | 4,975 | 19,931 | 24,906 | 20% |
| Any Drug | 63,550 | 170,488 | 234,038 | 27% |
| HCTZ | | 543 | 662 | 18% |
| NONTHIAZ | 1,018 | 1,948 | 2,966 | 34% |
| ACE | 321 | 1,602 | 1,923 | 17% |
| ARB | 213 | 1,363 | 1,576 | 14% |
| BETA | 1,771 | 7,712 | 9,483 | 19% |
| CCB | 460 | 1,541 | 2,001 | 23% |
| totals | 72,427 | 205,128 | 277,555 | |

Table 6 presents the incidence of edema when patients taking CELEBREX® alone, CELEBREX®+ any other drug, CELEBREX®+ various classes of anti-hypertensive. For this group, the patients are on CELEBREX® for more than 180 days.

TABLE 6
Incidence of edema, patients on celecoxib more than 180 days.

| #Pts | Edema | No Edema | Total Pts # | % Edema |
|---|---|---|---|---|
| Alone | 4975 | 19931 | 24906 | 20% |
| Any Drug | 23264 | 44752 | 68016 | 34% |
| HCTZ | 21 | 103 | 124 | 17% |
| NONTHIAZ | 307 | 228 | 535 | 57% |
| ACE | 59 | 272 | 331 | 18% |
| ARB | 37 | 254 | 291 | 13% |
| BETA | 491 | 1708 | 2199 | 22% |
| CCB | 126 | 306 | 432 | 29% |
| Total | 29280 | 67554 | 96834 | |

### Example 8

This application seeks the treatment of patients at target celecoxib AUC of 3400 ng*hr/mL (Mean AUC of 100 mg dose) or celecoxib AUC of 6800 ng*hr/mL (Mean AUC of 200 mg dose). Celecoxib AUC can be determined for example by LC/MS method with blood samples at various time points over at 48 hr period. There is no known method of predicting celecoxib AUC—therefore actual celecoxib AUC determination for each patient is required. The target AUCs were determined as the means of all available PK data for celecoxib at 100 mg and 200 mg dose levels as shown in Example 2.

TABLE 7

| | 100 mg | 200 mg |
|---|---|---|
| # of Values | 4 | 8 |
| Min. | 2465 | 5972 |
| Med. | 3395 | 6444 |
| Max. | 3418 | 6791 |
| Std. dev. | 859.9 | 831 |
| Std. error of mean | 429.9 | 293.8 |

### Example 9

Dissolution testing very widely used in formulation development, in monitoring the manufacturing process and as a quality control test. It can also be used to predict the in vivo performance of certain products. Dissolution testing has been successfully used for development and approval of generic solid oral dosage forms. Most recently, the use of dissolution testing has been extended to other solid generic dosage forms. Further, dissolution testing plays significant role in identifying the need for additional bioequivalence studies.

Dissolution testing should be carried out under physiological conditions, if possible. This allows interpretation of dissolution data with regard to in vivo performance of the product. However, strict adherence to the gastrointestinal environment need not be used in routine dissolution testing. The testing conditions should be based on physicochemical characteristics of the drug substance and the environmental conditions the dosage form might be exposed to after oral administration.

The volume of the dissolution medium is generally 500, 900, or 1000 mL. Sink conditions are desirable but not mandatory. An aqueous medium with pH range 1.2 to 6.8 (ionic strength of buffers the same as in USP) should be used. To simulate intestinal fluid (SIF), a dissolution medium of pH 6.8 should be employed. A higher pH should be justified on a case-by-case basis and, in general, should not exceed pH 8.0. To simulate gastric fluid (SGF), a dissolution medium of pH 1.2 should be employed without enzymes. The need for enzymes in SGF and SIF should be evaluated on a case-by-case basis and should be justified. Recent experience with gelatin capsule products indicates the possible need for enzymes (pepsin with SGF and pancreatin with SIF) to dissolve pellicles, if formed, to permit the dissolution of the drug. Use of water as a dissolution medium also is discouraged because test conditions such as pH and surface tension can vary depending on the source of water and may change during the dissolution test itself, due to the influence of the active and inactive ingredients. For water insoluble or sparingly water soluble drug products, use of a surfactant such as sodium lauryl sulfate is recommended (Shah 1989, 1995). The need for and the amount of the surfactant should be justified. Use of a hydro alcoholic medium is discouraged.

All dissolution tests for IR dosage forms should be conducted at 37±0.5° C. The basket and paddle method can be used for performing dissolution tests under multimedia conditions (e.g., the initial dissolution test can be carried out at pH 1.2, and, after a suitable time interval, a small amount of buffer can be added to raise pH to 6.8). Alternatively, if addition of an enzyme is desired, it can be added after initial studies (without enzymes). Certain drug products and formulations are sensitive to dissolved air in the dissolution medium and will need de-aeration. In general, capsule dosage forms tend to float during dissolution testing with the paddle method. In such cases, it is recommended that a few turns of a wire helix (USP) around the capsule be used.

The apparatus suitability tests should be carried out with a performance standard (i.e., calibrators) at least twice a year and after any significant equipment change or movement. However, a change from basket to paddle or vice versa may need recalibration. The equipment and dissolution methodology should include the product related operating instructions such as de-aeration of the dissolution medium and use of a wire helix for capsules. Validation of automated procedures compared to the manual procedures should be well documented. Validation of determinative steps in the dissolution testing process should comply with the set standards for analytical methodology.

In general, mild agitation conditions should be maintained during dissolution testing to allow maximum discriminating power and to detect products with poor in vivo performance. Using the basket method, the common agitation (or stirring speed) is 50-100 rpm; with the paddle method, it is 50-75 rpm.

Validation of the dissolution apparatus/methodology should include (1) the system suitability test using calibrators; (2) d-aeration, if necessary; (3) validation between manual and automated procedures; and (4) validation of a determinative step (i.e., analytical methods employed in quantitative analysis of dissolution samples). Under appropriate test conditions, a dissolution profile can characterize the product more precisely than a single point dissolution test. A dissolution profile comparison between pre-change and post-change products for SUPAC related changes, or with different strengths, helps assure similarity in product performance and signals bio-inequivalence.

Among several methods investigated for dissolution profile comparison, f2 is the simplest. Moore and Flanner proposed a model independent mathematical approach to compare the dissolution profile using two factors, f1 and f2 where Rt and Tt are the cumulative percentage dissolved at each of the selected n time points of the reference and test product respectively. (Moore & Flanner, Mathematical comparison of dissolution profiles. Pharma Tech. 20:64-74 (1996)). The factor f1 is proportional to the average difference between the two profiles, whereas factor f2 is inversely proportional to the average squared difference between the two profiles, with emphasis on the larger difference among all the time-points. The factor f2 measures the closeness between the two profiles.

Because of the nature of measurement, f1 was described as difference factor, and f2 as similarity factor. In dissolution profile comparisons, especially to assure similarity in product performance, regulatory interest is in knowing how similar the two curves are, and to have a measure which is more sensitive to large differences at any particular time point. For this reason, the f2 comparison has been the focus in Agency guidance.

When the two profiles are identical, f2=100. An average difference of 10% at all measured time points results in a f2 value of 50. FDA has set a public standard of f2 value between 50-100 to indicate similarity between two dissolution profiles.

For a dissolution profile comparison:

At least 12 units should be used for each profile determination. Mean dissolution values can be used to estimate the similarity factor, f2. To use mean data, the % coefficient of variation at the earlier point should not be more than 20% and at other time points should not be more than 10%. For circumstances where wide variability is observed, or a statistical evaluation of f2 metric is desired, a bootstrap approach to calculate a confidence interval can be performed.

The dissolution measurements of the two products (test and reference, pre- and post-change, two strengths) should be made under the same test conditions. The dissolution time points for both the profiles should be the same, e.g., for immediate release products 15, 30, 45 and 60 minutes, for extended release products 1, 2, 3, 5 and 8 hours.

Because f2 values are sensitive to the number of dissolution time points, only one measurement should be considered after 85% dissolution of the product. For products which are rapidly dissolving, i.e., more than 85% in 15 minutes or less, a profile comparison is not necessary.

The f2 comparison metric with a value of 50 or greater is a conservative, but a reliable estimate to assure product sameness and product performance. A f2 value of 50 or greater (50-100) ensures sameness or equivalence of the two curves and, thus, the performance of the two products.

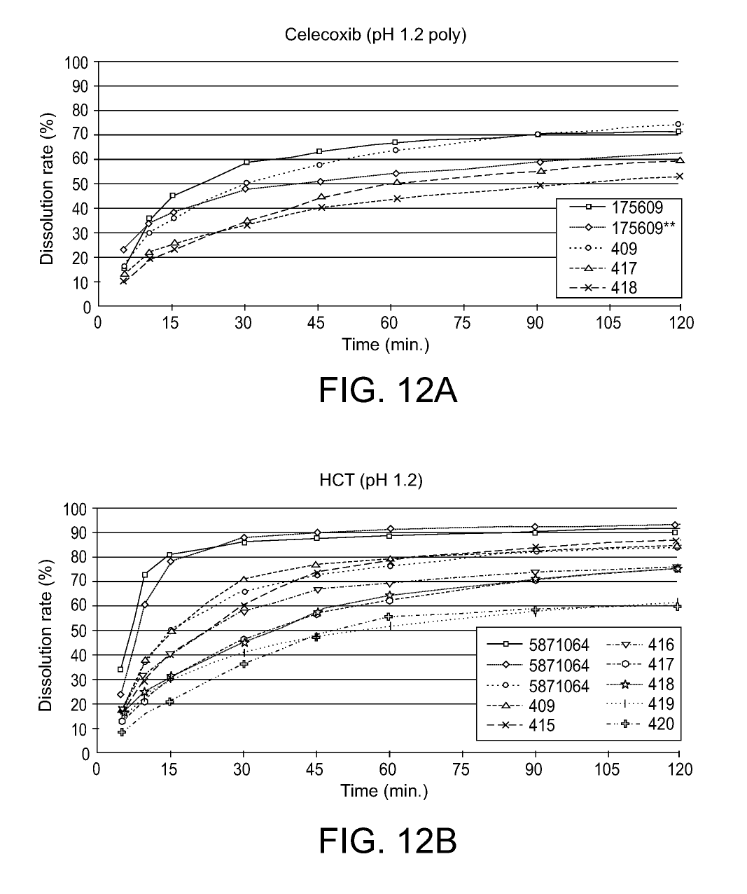

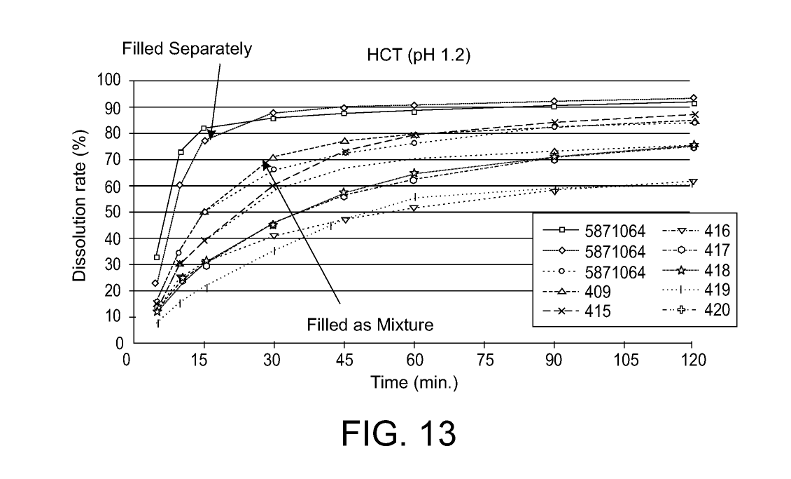

Surprising and unexpected we discovered that that celecoxib interferes with dissolution of hydrochlorothiazide in the art recognized dissolution assays using USP dissolution methods for pH 1.2, 4.0, and 6.8. When HCTZ and celecoxib were filled as mixture into a capsule, the dissolution of HCTZ, at pH 1.2, (FIG. 12) was too low for acceptance as equivalence to HCTZ as single agent in the reference listed drug (RLD). This was solved by filling HCTZ and celecoxib sequentially and separately into a single capsule. For example, the following figures shows how capsules in which hydrochlorothiazide and celecoxib are mixed show significantly lower dissolution of hydrochlorothiazide than capsules in which the two components have filled separately (FIG. 13).

Simple separation of the two drug substance by sequential and separate filling capsules with celecoxib followed by hydrochlorothiazide produced a marked improvement in the hydrochlorothiazide dissolution profiles.

Testing this formulation in pH 1.2, pH 4.0, pH 6.8, and water dissolution systems all resulted in hydrochlorothiazide dissolution profiles very similar to that found that for hydrochlorothiazide in single agent compositions. However, this simple separation of the two drug substance was not enough since suppression continued in pill in pill, capsule in pill and bilayer tablet, thus requiring formulation with excipient to counteract the dissolution suppression of HCTZ by celecoxib.

### Example 10

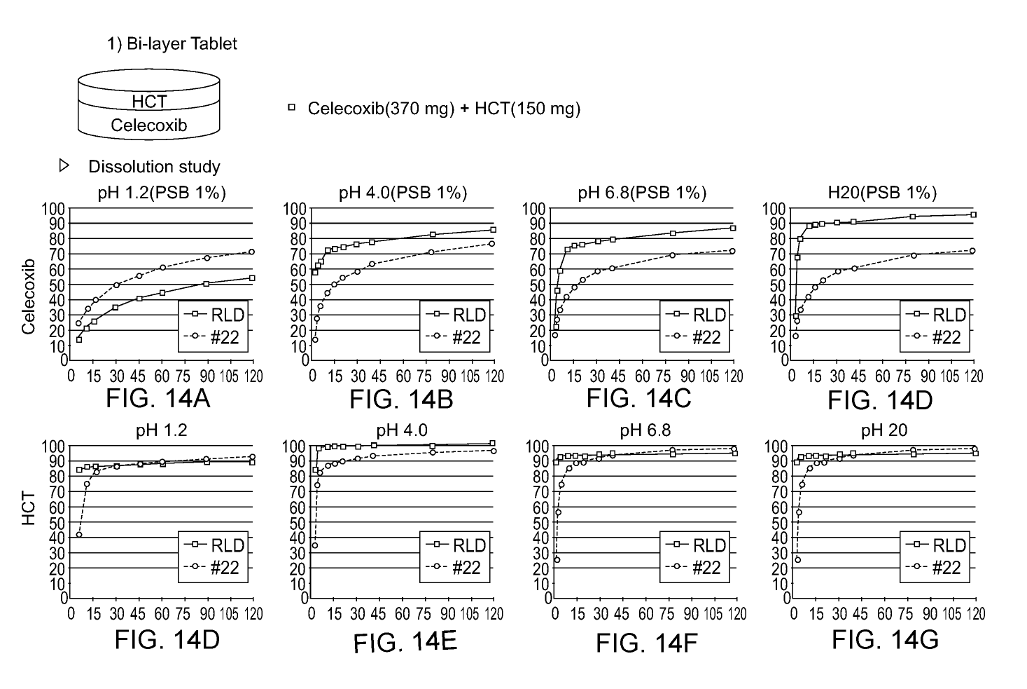

Surprisingly, celecoxib interferes with the dissolution of hydrochlorothiazide in art recognized dissolution assays, as described in Example 9, using USP dissolution methods for pH 1.2 (FIG. 14). Also surprisingly, celecoxib interference with HCTZ dissolution can be virtually eliminated by filling HCTZ and celecoxib sequentially and separately into a single capsule (FIG. 14). Additional studies were undertaken to confirm celecoxib's interference with the dissolution of HCTZ, determine the scope of this effect, and find solutions to it. Additional formulations were prepared which are set forth in Table 8.

TABLE 8
New formulations for capsule containing both HCTZ and Celecoxib.

| Formulation | #09 | #15 | #16 | #17 | #18 | #19 | #20 |
|---|---|---|---|---|---|---|---|
| Celecoxib | 200 | 200 | 200 | 200 | 200 | 200 | 200 |
| HCT | 25 | 25 | 25 | 25 | 25 | 25 | 25 |
| Lactose | 20 | 20 | 20 | 20 | 20 | 20 | 45 |
| Povidone | 6.8 | 13 | 7 | 16 | 16 | 7 | 7 |
| Colloidal silicon | 4 | 4 | 5.5 | 4 | 4 | 2 | 2 |
| dioxide | | | | | | | |
| SLS | 8.1 | 16 | 8 | 16 | 8 | 8 | 8 |
| Ac-Di-Sol (Pre-Mix) | — | 4 | — | 4 | 4 | — | — |
| Ac-Di-Sol (Post-Mix) | 8 | 8 | 14 | 8 | 8 | 8 | 8 |
| Mg-st | 2.3 | 2 | 2.5 | 2 | 2 | 2 | 2 |
| Total | 274.2 | 292 | 282 | 295 | 287 | 272 | 297 |
| Cap. Size | #1 | #1 | #1 | #1 | #1 | #1 | #1 |
| Comment | Colloidal | Increase | Increase | Increase | Reduce SLS | After putting | Increase |
| | silicon | binder | Ac-Di-Sol | Binder | | in a mixed | Filler |
| | dioxide | and the | Post Mix | Celecoxib (D90: 14 um_Mylan) | | HCT | |
| | added at | surfactant | | | | | |
| | pre-mixing | amount in | | | | | |
| | | order to | | | | | |
| | | increase | | | | | |
| | | water | | | | | |
| | | absorption | | | | | |

HCT (D90: 45 um_polpharma)

The HCTZ/celecoxib capsule formulations of Table 8 were tested against the appropriate RLDs (Table 9). At pH 1.2 the dissolution of HCTZ was suppressed in all formulations.

TABLE 9
Capsule Dissolution Test Results

| | | | | Time (min) | | | | | | | |
|---|---|---|---|---|---|---|---|---|---|---|---|
| | Medium | | | 5 | 10 | 15 | 30 | 45 | 60 | 90 | 120 |
| Cele | pH 1.2 | RLD | J75609 | 14 | 22 | 26 | 34 | 40 | 44 | 49 | 54 |
| | poly | Test | #09 | 11 | 19 | 23 | 35 | 44 | 50 | 56 | 60 |
| | | | #15 | | | | | | | | |
| | | | #16 | | | | | | | | |
| | | | #17 | 16 | 34 | 45 | 58 | 63 | 67 | 70 | 72 |
| | | | #18 | 17 | 30 | 36 | 50 | 58 | 63 | 70 | 74 |
| | | | #19 | | | | | | | | |
| | | | #20 | | | | | | | | |
| HCT | pH 1.2 | RLD | 887106A | 41 | 74 | 82 | 86 | 87 | 88 | 90 | 92 |
| | | Test | #09 | 14 | 31 | 39 | 60 | 73 | 79 | 84 | 87 |
| | | | #15 | 11 | 23 | 30 | 46 | 56 | 62 | 71 | 75 |
| | | | #16 | 12 | 24 | 30 | 46 | 58 | 64 | 71 | 75 |
| | | | #17 | 17 | 37 | 50 | 66 | 72 | 77 | 83 | 84 |
| | | | #18 | 14 | 26 | 31 | 42 | 48 | 52 | 58 | 62 |
| | | | #19 | 8 | 16 | 22 | 36 | 48 | 56 | 59 | 61 |
| | | | #20 | 13 | 31 | 40 | 58 | 67 | 70 | 73 | 76 |

As an alternative to capsules, a bilayered tablet formulation was prepared (Table 10).

TABLE 10
Bilayered Tablet Formulation

| | | mg/ | | | mg/ |
|---|---|---|---|---|---|
| | Celecoxib | Tab. | | HCT | Tab. |
| Binder | Povidone(PVPK-30) | 5 | Direct | Hydrochlo- | 25 |
| Layer | | | compression | rothiazide | |
| between | SLS | 6 | | Supertab | 108.5 |
| the two | | | | 30GR | |
| layers | | | | | |
| Pre | Celecoxib | 200 | | Aerosil 200 | 4 |
| mixing | Aerosil 200 | 3 | | Ac-Di-sol | 10 |
| | Supertab 30GR | 70 | | Ferric oxide | 0.5 |
| | | | | red | |
| Post | Aerosil 200 | 2 | | Mg stearate | 2 |
| mixing | Ac-Di-sol | 8 | | | |
| | Mg stearate | 6 | | | |
| Total | | 300 | Total | | 150 |

When the bilayer tablet was dissolution tested, celecoxib was suppressed at pH 4.0 and normal at pH 1.2. But, HCTZ dissolution was recovered at both pH 1.2 and pH.2. (see Table 11).

TABLE 11
Tablet Dissolution Test Results

| | | | | Time (min) | | | | | | | |
|---|---|---|---|---|---|---|---|---|---|---|---|
| | Medium | | | 5 | 10 | 15 | 30 | 45 | 60 | 90 | 120 |
| Cele | pH 1.2 | RLD | J75609 | 14 | 22 | 26 | 34 | 40 | 44 | 49 | 54 |
| | poly | Test | #21 | 10 | 20 | 24 | 30 | 34 | 37 | 41 | 45 |
| | pH 4.0 | RLD | J75609 | 58 | 61 | 63 | 72 | 73 | 74 | 76 | 77 |
| | poly | Test | #21 | 12 | 27 | 31 | 37 | 41 | 44 | 48 | 51 |
| HCT | pH 1.2 | RLD | 887106A | 41 | 74 | 82 | 86 | 87 | 88 | 90 | 92 |
| | | Test | #21 | 44 | 65 | 75 | 81 | 82 | 83 | 85 | 85 |
| | pH 4.0 | RLD | 887106A | 34 | 73 | 82 | 86 | 88 | 89 | 91 | 92 |
| | | Test | #21 | 78 | 85 | 85 | 85 | 86 | 86 | 87 | 88 |

From these results, the prior art describing the combination of HCTZ and celecoxib, none of which have been reduced to practice, did not anticipate the HCTZ and celecoxib dissolution interferences observed in both tablet and capsule presentations. Because dissolution is likely to be predictive of absorption of the drugs in humans, dissolution interferences represent a serious problem that was not appreciated by the art.

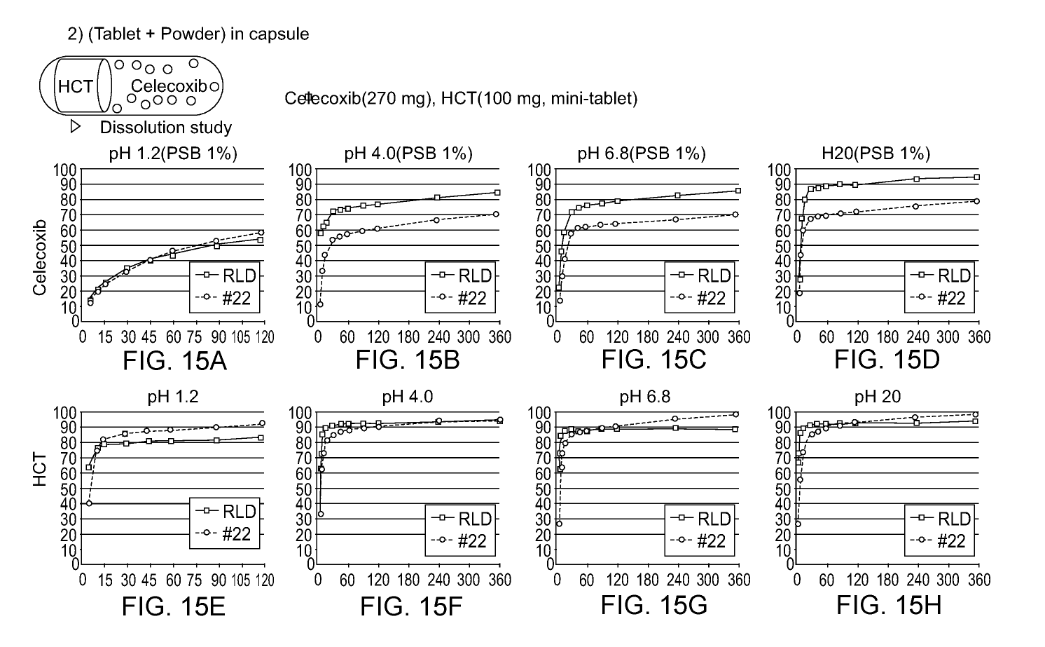

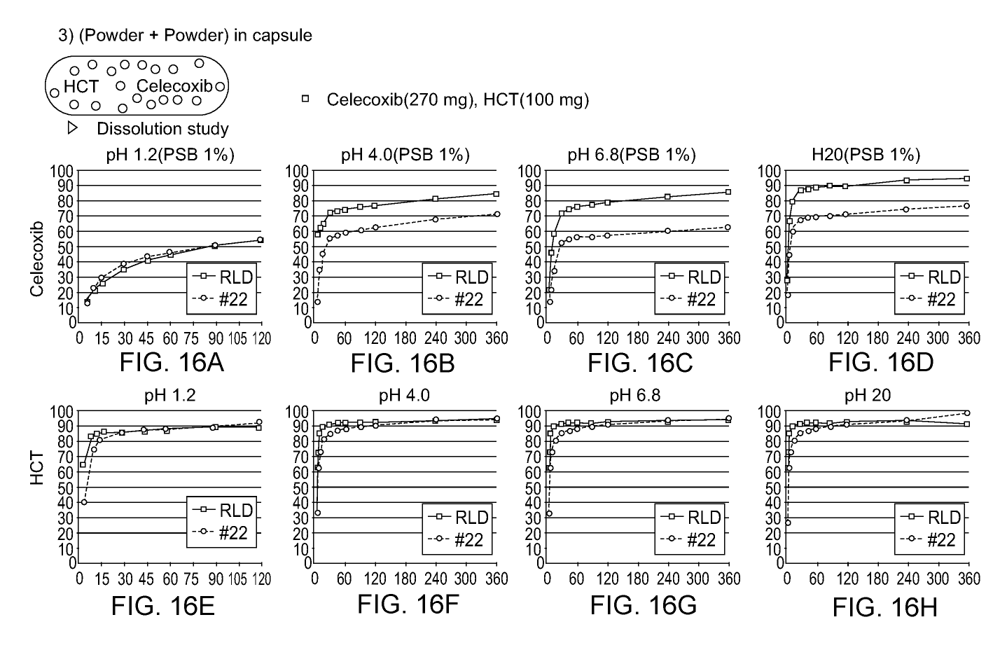

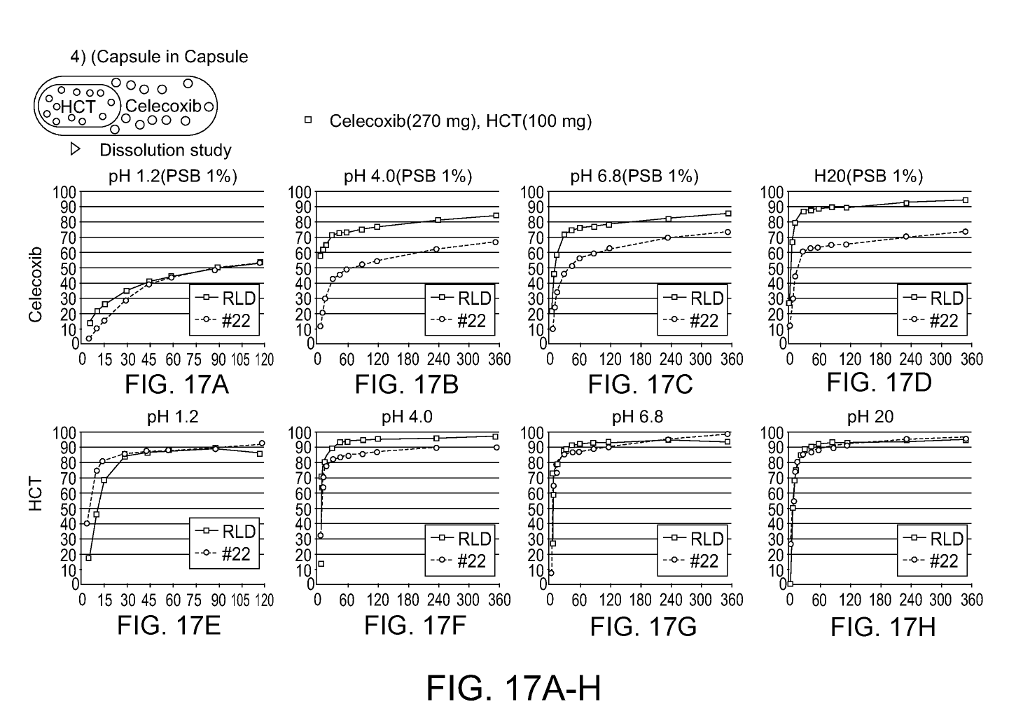

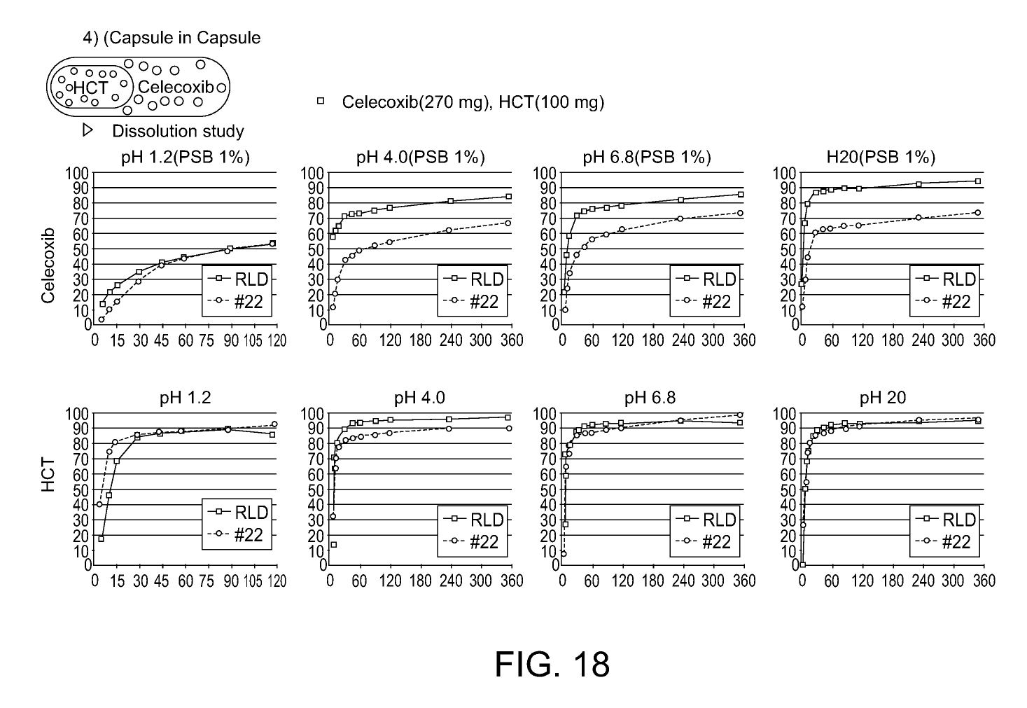

To remedy the problem presented by the interference of HCTZ and celecoxib dissolution, multiple presentations of celecoxib plus HCTZ fixed dose combination (FDC) underwent dissolution testing. Dissolution profiles similar to that of HCTZ in a single agent dosage form were obtained only when there was a physical separation between celecoxib and HCTZ. However, this separation alone was not sufficient as celecoxib dissolution in the HCTZ+celecoxib FDC at pH 4.0, pH 6.8, and H₂O was impaired in all formulations examined. Exemplary formulations are shown in FIG. 15.

As stated above, the solution to the dissolution interference problem is, most often, the separation of HCTZ and celecoxib (including, but are not limited to, i.e., a bilayer tablet, a capsule with separate and sequential filling, a capsule in a capsule or a tablet or minitablets in a capsule.) Additionally, to keep the FDC dissolution profiles matching those of CELEBREX® and HCTZ RLDs, disintegrants were required in the celecoxib portion of the formulation but not in the HTCZ portion. Suitable disintegrants include, but are not limited to, povidone (for example, KOLIDONE™), crospovidone (for example, POLYPLASDONE™), croscarmellose sodium, carboxymethylcellulose calcium and L-HPC. The resulting dissolution of celecoxib matched that of the RLD when the disintegrant was added to celecoxib (but not to HTCZ) (See Table 12).

TABLE 12

| Cele | Medium | Time (min) | | 10 | 20 | 30 | 45 | 60 |
|---|---|---|---|---|---|---|---|---|
| | pH 12 + | RLD | J75609 | 56 | 78 | 82 | 85 | 86 |
| | 1% SLS | Test | #29 | 52 | 68 | 75 | 78 | 80 |
| | Medium | Time (min) | | 5 | 10 | 15 | 30 | 45 |
| | pH 1.2 | RLD | J75609 | 14 | 22 | 26 | 34 | 40 |
| | poly | Test | #29 | 17 | 27 | 32 | 42 | 47 |
| | | | #32 | 14 | 22 | 25 | 32 | 37 |
| | pH 4.0 | RLD | C140232 | 11 | 38 | 53 | 62 | 65 |
| | poly | Test | #29 | 16 | 33 | 40 | 47 | 50 |
| | pH 6.8 | RLD | C140232 | 9 | 40 | 57 | 75 | 81 |
| | poly | Test | #29 | 16 | 39 | 49 | 62 | 69 |
| | H2O | RLD | J75609 | 27 | 67 | 80 | 87 | 88 |
| | poly | Test | #29 | 38 | 61 | 77 | 87 | 89 |

The dissolution interference disclosed herein may also be reflected by poor bioavailability of hydrochlorothiazide when given orally to patients. Accordingly, dosage forms which are simple mixture of celecoxib and HCTZ should be avoided. Dissolution should be monitored and controlled to ensure that HCTZ dissolution is not inhibited by celecoxib. FDC with separation of two components should only be used with a disintegrant in the celecoxib portion as shown herein. Disintegrants, an important excipient of the tablet formulation, are added to tablets to induce breakup of tablet when it comes in contact with aqueous fluid. This process of desegregation of constituent particles before the drug dissolution occurs is known as disintegration process and excipients which induce this process are known as disintegrants. The objectives behind addition of disintegrants are to increase surface area of the tablet fragments and to overcome cohesive forces that keep particles together in a tablet.

Exemplary disintegrants include, e.g., L-HPC (low substituted hydroxypropyl cellulose), other disintegrants include povidone (for example, Kolidone™, etc.), crospovidone (for example, Polyplasdone™, etc.), croscarmellose sodium, and carboxymethylcellulose calcium, as well as modified starches and modified cellulose. Other disintegrants include those listed in the tables below (see also AAPS PharmSciTech. 2012 December; 13(4): 1054-1062., Published online 2012 Aug. 17. doi: 10.1208/s12249-012-9835-y.).

Starch was the first disintegrating agent widely used in tablet manufacturing. Before 1906 potato starch and corn starch were used as disintegrants in tablet formulation. However, native starches have certain limitations and have been replaced by certain modified starches with specialized characteristics.

The mechanism of action of starch is wicking and restoration of deformed starch particles on contact with aqueous fluid and in doing so release of certain amount of stress which is responsible for disruption of hydrogen bonding formed during compression.

Lowenthal & Wood proved that the rupture of the surface of a tablet employing starch as disintegrant occurs where starch agglomerates were found. The conditions best suited for rapid tablet disintegration are sufficient number of starch agglomerates, low compressive pressure and the presence of water.

The concentration of starch used is also very crucial part. If it is below the optimum concentration, then there are insufficient channels for capillary action and if it is above optimum concentration then it will be difficult to compress the tablet.

Pregelatinized starch is produced by the hydrolyzing and rupturing of the starch grain. It is a directly compressible disintegrants and its optimum concentration is 5-10%. The main mechanism of action of Pregelatinized starch is through swelling.

To have a high swelling properties and faster disintegration, starch is modified by carboxy methylation followed by cross linking, which is available in market as cross linked starch. One of them is SODIUM STARCH GLYCOLATE. Even low substituted carboxymethyl starches are also marketed as ExplotabO and Primojel(r).

Mechanism of action of these modified starches are rapid and extensive swelling with minimum gelling. And its optimum concentration is 4-6%. If it goes beyond its limit, then it produces viscous and gelatinous mass which increases the disintegration time by resisting the breakup of tablet. They are highly efficient at low concentration because of their greater swelling capacity.

Sodium carboxy methylcellulose (NaCMC and CARMELLOSE sodium) has highly hydrophilic structure and is soluble in water. But when it is modified by internally crosslinking we get modified crosslinked cellulose i.e. Crosscarmellose sodium which is nearly water insoluble due to cross linking. It rapidly swells to 4-8 times its original volume when it comes in contact with water.

Microcrystalline cellulose (MCC). MCC exhibit very good disintegrating properties because MCC is insoluble and act by wicking action. The moisture breaks the hydrogen bonding between adjacent bundles of MCC. It also serves as an excellent binder and has a tendency to develop static charges in the presence of excessive moisture content. Therefore, sometimes it causes separation in granulation. This can be partially overcome by drying the cellulose to remove the moisture. Alginates are hydrophilic colloidal substances which has high sorption capacity. Chemically, they are alginic acid and salts of alginic acid. Alginic acid is insoluble in water, slightly acidic in reaction. Hence, it should be used in only acidic or neutral granulation. Unlike starch and MCC, alginates do not retard flow and can be successfully used with ascorbic acid, multivitamin formulations and acid salts of organic bases.

Ion exchange resin (AmbreliteO IPR-88) has highest water uptake capacity than other disintegrating agents like starch and Sodium CMC. It has tendency to adsorb certain drugs. This miscellaneous category includes disintegrants like surfactants, gas producing disintegrants and hydrous aluminium silicate. Gas producing disintegrating agents is used in soluble tablet, dispersible tablet and effervescent tablet.

PolyplasdoneOXL and PolyplasdoneOXL10 act by wicking, swelling and possibly some deformation recovery. Polyplasdone(r)XL do not reduce tablet hardness, provide rapid disintegration and improved dissolution. Polyplasdone(r) as disintegrating agent has small particle size distribution that impart a smooth mouth feel to dissolve quickly. Chewable tablet does not require addition of disintegrant.

| | CONCENTRATION IN | |
|---|---|---|
| DISINTEGRANTS | GRANULES (% W/W) | SPECIAL COMMENTS |
| Starch USP | 5-20 | Higher amount is required, |
| | | poorly compressible |
| Starch 1500 | 5-15 | — |
| Avicel(r)(PH 101, PH 102) | 10-20 | Lubricant properties and |
| | | directly compressible |
| Solka floc(r) | 5-15 | Purified wood cellulose |
| Alginic acid | 1-5 | Acts by swelling |
| Na alginate | 2.5-10 | Acts by swelling |
| Explotab(r) | 2-8 | Sodium starch glycolate, |
| | | superdisintegrant. |
| Polyplasdone(r)(XL) | 0.5-5 | Crosslinked PVP |
| Amberlite(r) (IPR 88) | 0.5-5 | Ion exchange resin |
| Methyl cellulose, Na CMC, | 5-10 | — |
| HPMC | | |
| AC-Di-Sol(r) | 1-3 | Direct compression |
| | 2-4 | Wet granulation |
| Carbon dioxide | — | Created insitu in effervescent |
| | | tablet |

| | | MECHANISM OF | SPECIAL |
|---|---|---|---|
| DISINTEGRANTS | EXAMPLE OF | ACTION | COMMENT |
| Crosscarmellose(r) | Crosslinked | Swells 4-8 folds in <10 | Swells in two |
| Ac-Di-Sol(r) | cellulose | seconds. | dimensions. |
| Nymce ZSX(r) | | Swelling and | Direct compression |
| Primellose(r) | | wicking both. | or granulation |
| Solutab(r) | | | Starch free |
| Vivasol(r) | | | |
| Crosspovidone | Crosslinked PVP | Swells very little | Water insoluble |
| Crosspovidon M(r) | | and returns to | and spongy in |
| Kollidon(r) | | original size after | nature so get porous |
| Polyplasdone(r) | | compression but act | tablet |
| | | by capillary action | |
| Sodium starch glycolate | Crosslinked starch | Swells 7-12 folds | Swells in three |
| Explotab(r) | | in <30 seconds | dimensions and |
| Primogel(r) | | | high level serve as |
| | | | sustain release |
| | | | matrix |
| Alginic acid NF | Crosslinked | Rapid swelling in | Promote |
| Satialgine(r) | alginic acid | aqueous medium or | disintegration in |
| | | wicking action | both dry or wet |
| | | | granulation |
| Soy polysaccharides | Natural super | | Does not contain |
| Emcosoy(r) | disintegrant | | any starch or sugar. |
| | | | Used in nutritional |
| | | | products. |
| Calcium silicate | | Wicking action | Highly porous, |
| | | | light weight |
| | | | optimum |
| | | | concentration is |
| | | | between 20-40% |

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

What is claimed is:

1. A composition for treating pain in a mammal, the composition comprising celecoxib and hydrochlorothiazide, wherein
(a) the celecoxib and the hydrochlorothiazide are in a single dosage unit;
(b) the celecoxib and the hydrochlorothiazide are each at a fixed dosage; where the HCTZ and the celecoxib are physically separated;
(c) the celecoxib is mixed with a disintegrant to counteract the dissolution suppression in the Fixed Dose Combination (FDC); and
(d) a dissolution profile for each of celecoxib and hydrochlorothiazide in the single dosage unit is equivalent to a dissolution profile of celecoxib and a dissolution profile hydrochlorothiazide if administered in separate dosage units, such that the f2 score based on this comparison is 50 to 100.

2. The composition of claim 1, wherein the single dosage unit has separate compartments for the celecoxib and the hydrochlorothiazide.

3. The composition of claim 1, wherein the pain is arthritic pain.

4. The composition of claim 1, wherein the arthritic pain is osteoarthritic.

5. The composition of claim 1, wherein the composition is in the form of a pill in a pill, a capsule in a capsule, a tablet in powder in a capsule, a mini-tablet in powder in a capsule, or a bilayer tablet.

6. The composition of claim 1, wherein the dose of celecoxib is 50 to 400 mg.

7. The composition of claim 1, where the dose of hydrochlorothiazide is 5 to 100 mg.

8. The composition of claim 1, wherein the dose of celecoxib is 100 mg and the dose of hydrochlorothiazide is 12.5 mg.

9. The composition of claim 1, wherein the dose of celecoxib is 200 mg and the dose of hydrochlorothiazide is 12.5 mg.

10. The composition of claim 1, wherein the dose of celecoxib is 100 mg and the dose of hydrochlorothiazide is 25 mg.

11. The composition of claim 1, wherein the dose of celecoxib is 200 mg and the dose of hydrochlorothiazide is 25 mg.

12. The composition of claim 1, wherein the dose of celecoxib is 400 mg and the dose of hydrochlorothiazide is 150 mg.

13. The composition of claim 1, wherein the dose of celecoxib is 300 mg and the dose of hydrochlorothiazide is 100 mg.

14. The composition of claim 1, wherein the disintegrant is selected from the group consisting of L-HPC, povidone, crospovidone, and carboxymethylcellulose calcium.

15. The composition of claim 1, wherein the disintegrant is L-HPC.

16. A method for the treatment of pain with a reduction in the incidence of edema, comprising the administration of the composition of claim 1 to a mammal.

17. The method of claim 16, where in the pain is arthritic pain.

18. The method of claim 17, wherein the arthritic pain is osteoarthritic pain.
